(19) United States
(12) Patent Application Publication
(10) Pub. No.: US 2022/0359083 A1
(43) Pub. Date: Nov. 10, 2022

(54) METHODS AND SYSTEMS FOR FACILITATING ASSESSMENT OF WELL-BEING OF A USER BASED ON FEEDBACK

(71) Applicants: Jorge Iriso, Oakland, CA (US); Aimee Beryl Cole, Oakland, CA (US); Aitor Iriso, Oakland, CA (US); Eneko Iriso, Oakland, CA (US)

(72) Inventors: Jorge Iriso, Oakland, CA (US); Aimee Beryl Cole, Oakland, CA (US); Aitor Iriso, Oakland, CA (US); Eneko Iriso, Oakland, CA (US)

(21) Appl. No.: 17/736,948

(22) Filed: May 4, 2022

Related U.S. Application Data
(60) Provisional application No. 63/184,028, filed on May 4, 2021.

(51) Int. Cl. G16H 50/30; G16H 50/20; G16H 10/20; G16H 10/60; H04L 9/00

(52) U.S. Cl. CPC G16H 50/30; G16H 50/20; G16H 10/20; G16H 10/60; H04L 9/50

(57) ABSTRACT

The present invention describes methods and systems for facilitating assessment of well-being of a user based on feedback. Further, the disclosed system may be configured for determining a Human Aspect Value (HAV) that may be a quantifiable metric an organization can utilize in real-time to assess its well-being from employee-driven feedback. The Human Aspect Value is the sum of the Aspect Value and the Participation (Engagement) Value multiplied by their respective coefficients or weights which are customizable depending on the organization's needs.

### FIELD OF THE INVENTION

The present invention relates generally to data processing. More specifically, the present invention is methods and systems for facilitating assessment of well-being of a user based on feedback.

### BACKGROUND OF THE INVENTION

The field of data processing is technologically important to several industries, business organizations, and/or individuals.

Existing techniques for facilitating assessment of well-being of a user based on feedback are deficient with regard to several aspects. For instance, current technologies do not facilitate fostering a connection and personal development of an individual (or an employee). Furthermore, current technologies do not determine a quantifiable metric (such as human aspect value) for facilitating assessing well-being of an individual. Moreover, current technologies do not facilitate the scheduling of an appointment for assisting the individual facing well-being issues.

Therefore, there is a need for improved methods and systems for facilitating assessment of well-being of a user based on feedback that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

According to some embodiments, a method for facilitating assessment of well-being of a user based on feedback is disclosed. Accordingly, the method may include receiving, using a communication device, a request from at least one first user device associated with at least one first user. Further, the method may include retrieving, using a storage device, a questionnaire from a blockchain. Further, the method may include transmitting, using the communication device, the questionnaire to at least one second user device. Further, the method may include receiving, using the communication device, at least one response associated with a time interval from the at least one second user device, wherein the at least one response is associated with the questionnaire. Further, the method may include storing, using the storage device, the at least one response on the blockchain. Further, the method may include retrieving, using the storage device, customizable individual coefficients (or weights) corresponding to the human categories of the questionnaire from the blockchain. Further, the method may include analyzing, using a processing device, the at least one response, the questionnaire based on the customizable individual coefficients. Further, the method may include generating, using the processing device, a graphical representation based on the analyzing. Further, the method may include retrieving, using the storage device, a participation value and a predetermined coefficient from the blockchain. Further, the method may include processing, using the processing device, the participation value, the customizable individual coefficients, the predetermined coefficient, the at least one response, and the questionnaire. Further, the method may include determining, using the processing device, human aspect value associated with the at least one second user based on the processing. Further, the method may include transmitting, using the communication device, the human aspect value and the graphical representation to at least one of the at least one first user device and the at least one second user device. Further, the method may include storing, using the storage device, the human aspect value and the graphical representation on the blockchain.

According to some aspects, a system for facilitating assessment of well-being of a user based on feedback is disclosed. Accordingly, the system may include a communication device configured for receiving a request from at least one first user device associated with at least one first user. Further, the communication device may be configured for transmitting a questionnaire to at least one second user device. Further, the communication device may be configured for receiving at least one response associated with a time interval from the at least one second user device, wherein the at least one response is associated with the questionnaire. Further, the communication device may be configured for transmitting a human aspect value and a graphical representation to at least one of the at least one first user device and the at least one second user device. Further, the system may include a processing device configured for analyzing the at least one response, the questionnaire based on customizable individual coefficients. Further, the processing device may be configured for generating the graphical representation based on the analyzing. Further, the processing device may be configured for processing a participation value, the customizable individual coefficients, a predetermined coefficient, the at least one response, and the questionnaire. Further, the processing device may be configured for determining the human aspect value associated with the at least one second user based on the processing. Further, the system may include a storage device configured for retrieving the questionnaire from a blockchain. Further, the storage device may be configured for storing the at least one response on the blockchain. Further, the storage device may be configured for retrieving the customizable individual coefficients (or weights) corresponding to the human categories of the questionnaire from the blockchain. Further, the storage device may be configured for storing the human aspect value and the graphical representation on the blockchain. Further, the storage device may be configured for retrieving the participation value and the predetermined coefficient from the blockchain.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 is a block diagram of a system for facilitating assessment of well-being of a user based on feedback, in accordance with some embodiments.

FIG. 3 is a flowchart of a method for facilitating assessment of well-being of a user based on feedback, in accordance with some embodiments.

FIG. 4 is a schematic of a system for facilitating assessment of well-being of a user based on feedback, in accordance with some embodiments.

FIG. 5 is a block diagram of a computing device for implementing the methods disclosed herein, in accordance with some embodiments.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header. The present disclosure includes many aspects and features. Moreover, while many aspects and features relate to, and are described in the context of methods and systems for facilitating assessment of well-being of a user based on feedback, embodiments of the present disclosure are not limited to use only in this context.

Overview:

The present disclosure describes methods and systems for facilitating assessment of well-being of a user based on feedback. Further, the disclosed system may be configured for determining a Human Aspect Value (HAV) that may be a quantifiable metric an organization can utilize in real-time to assess its well-being from employee-driven feedback. The Human Aspect Value is the sum of the Aspect Value and the Participation (Engagement) Value multiplied by their respective coefficients or weights which are customizable depending on the organization's needs.

Further, in an instance, the default assessment may be a 60/40 ratio between aspect value and participation value.

Total Weight=1.0

Human Aspect Value=(0.6) (Aspect Value)+(0.4) (Participation (Engagement) Value)

Further, users may be asked daily a minimum of 4 questions from 4 Human categories or aspects: mind, body, social, environment (rest are customizable) offered via a mobile interface and/or desktop for user input. Further, the users may respond to the questions whenever they like by selecting the answer that best captures their self-evaluation which corresponds to a value in the range of 1-5 (worst-best), and have the ability to re-evaluate anytime. Further, the responses may be recorded throughout the day and the final or most recent set of responses are saved to our database at the end of the day before the refreshing for the following day. Further, using a data set of responses collected over a week(minimum) or a month(recommended) but not limited to any time frame, values collected are then averaged using customizable individual coefficients or weights depending on how the aspects are valued by the organization. For instance, construction companies may value body responses more than a software firm, for instance, default configuration:

Total weight=1.0
0.25×Mind values (1-5)
0.25×Body values (1-5)
0.25×Social values (1-5)
0.25×Environment values (1-5)

Since, construction company example is body intensive:

Total weight=1.0
0.3×Mind values (1-5)
0.5×Body values (1-5)←more valued (heavier weight)
0.1×Social values (1-5)
0.1×Environment values (1-5)

Further, average weighted values may be then plotted in relation to a status scale from 1-5 and ranked as a final Aspect Value for the selected time period. Aspect Value ranges i.e. are as follows*: *value descriptors are configurable
0-1: Very Poor
1-2: Poor
2-3: Adequate
3-4: Good
4-5: Excellent

Further, the final Aspect Value may be then multiplied by a customizable coefficient before being summed with the participation value and its respective coefficient. For instance (Default):

Total Weight=1.0

Human Aspect Value=(0.6) (Aspect Value)+(0.4) (Participation Value)

Further, the Participation (Engagement) Value measures contribution of user input based on a configurable data set comprising but not limited to: Aspect Value responses (4 Aspects minimum) as well as the contribution of user input from a set of interactive responses (or applications). Both the application and aspect contributions are then weighted and summed based on how the organization decides to configure the equation.

For instance:

Participation (Engagement) Value may be calculated as:

Total Weight=1.0

Participation Value=(0.6) Application Contribution+(0.4) Aspect Contribution

Further, after recording the responses from the 4 daily Aspect questions for the data period selected(week or month minimum), the contribution is then counted from the same period, the Aspect Value is calculated as a percentage of responses submitted by a user divided by the total number of responses that user can submit (4 per day). Further, the percentage may be then multiplied by 5 resulting in the aspect contribution before it gets evaluated in the final equation.

For instance:

Aspect Contribution: (1-week period)

Responses recorded/total=14/15=93.3%

Aspect Contribution=5×93.3%=4.67×(weight)

In addition to the aspect contribution, the Application contribution may be calculated. Further, the applications may include but are not limited to: Questions, Decisions, Ideas, Achievements (default configuration) that record values (0-5) depending on how many times the user contributes; 0 being the lowest (no contribution) and 5 (max contribution) per application for the period.

Further, each application may be weighed individually out of a total weight of 1.0 before the application contribution is weighed as a whole.

For instance:

Application Contribution: (same period)

Total Weight=1.0

### User Contribution Example

(0.25) Questions (3 times)+(0.25) Decisions (5 times)+(0.25) Ideas (2 times)+(0.25) Achievements (1 time)

=(0.25)3+(0.25)5+(0.25)2+(0.25)1

=0.75+1.25+0.5+0.25

=2.75×(weight)

Further, the Participation (Engagement) Value may be calculated as the sum of the weighted and computed Aspect and Application contributions as shown below.

For instance:

(Default Configuration)

Total Weight=1.0

Participation Value=(0.6) (2.75)+(0.4) (4.67)←from examples above

=1.65+1.868

=3.51

Further, unlike the aspect value that may be plotted on a fixed range from 1-5, the

participation value generated is only compared to the average participation value of all other users and a curve is applied.

Further, the Aspect Value may be obtained from the period (4.33 may be used which correlates to an “excellent” value). Further, the Participation (Engagement) Value may be obtained for the same period as well (3.51 may be used from the example prior). Further, the participation (Engagement) is based on the curve of all users.

Further, the Human Aspect Value (HAV) may be calculated using the equation:

(Default Configuration)

Human Aspect Value:

Total Weight=1.0

HAV=(0.6) (Aspect Value)+(0.4) (Participation (Engagement) Value)

HAV=(0.6) (4.33)+(0.4) (3.51)

HAV=4.002

Further, the HAV result may be evaluated by referring to the traditional status scale (1-5):
0-1: Very Poor
1-2: Poor
2-3: Adequate
3-4: Good
4-5: Excellent

Further, the HAV of 4.002 indicates that the status is “excellent” and the users evaluated appear to be in good spirits according to their Aspect Value. Also, their contributions measured by their Participation (Engagement) appear to be adequate although those values can vary based on the curve if Participation is evaluated solely. Further, the disclosed system may be associated with a software platform (such as the software application and a website). Further, the disclosed system may be configured for allowing the user to submit a message with the highest of the priorities to an official (such as a human resource manager). Further, the user clicks an SOS button and enters a brief reason in the text field. Further, the text field may include dropdowns. Further, the alert arrives at the recipient based on configuration. Further, a custom in-person reply may be sent to the user (or originating user). Further, based on the selection of the pulldown menu, an automated response may be generated.

Further, the data captured may be analyzed and suggestions may be presented to the admins based on default parameters. Further, the disclosed system may be configured for delivering individualized responses. For instance: similar responses on the same weekday(s) may prompt response i.e. “you have the same or similar response on the same day(s), can you think of a reason (mind—why your motivation level is similar on XYZ parameter), (body—why your physical condition level is similar on XYZ parameter), (social—why your social response level is similar on XYZ parameter).

Further, the software application may alert the user when there are new messages, opinion, decisions or any other action that require his attention. Further, admin/Global/Dept/Group creates a notification for delivery to all/specific groups. Further, alert arrives on landing when opening the software application may have “read & Understood” acknowledgment, save for later, selection buttons, etc.

Further, based on the analyzed data and patterns, the software application may present automated prompts. Depending on a predetermined timeline, a user(s) is sent a Question/Prompt for a response, etc. (could be used for recently onboardedee's, after initiatives, etc.). Further, the user responds to the prompt via selection or an individualized response report can be generated with the responses for data.

Further, the software application may allow the recording of video and pictures to go along with any message, question, or decision. Further, the sender records a short message—may be included with the survey/questionnaire. Further, a recipient completes a survey/questionnaire. Further, the results may be measured. Further, the result may be associated with better engagement due to personal message context.

Further, the software application may present informational announcements, news, alerts, etc. Further, the announcements may be sent on the fly or scheduled through the admin dashboard. Further, the announcements are viewed by the users and can be archived to view again up to a certain time frame (configurable).

Further, the software application may display the automated prompt remembering the user to complete questions, decisions, or any other unanswered action. Further, a reminder may be sent with a link to a prior message. Further, the disclosed system may be configured for determining the users that may not have responded/finished. Further, the reports are available to see completion. Further, red flag departments/groups may be associated with low response.

Further, the software application may allow capturing user information that may be displayed to other users. Further, the each user may complete a profile that may include personal info that may be of interest to their colleagues/manager. Further, the disclosed system may foster connection and personal development.

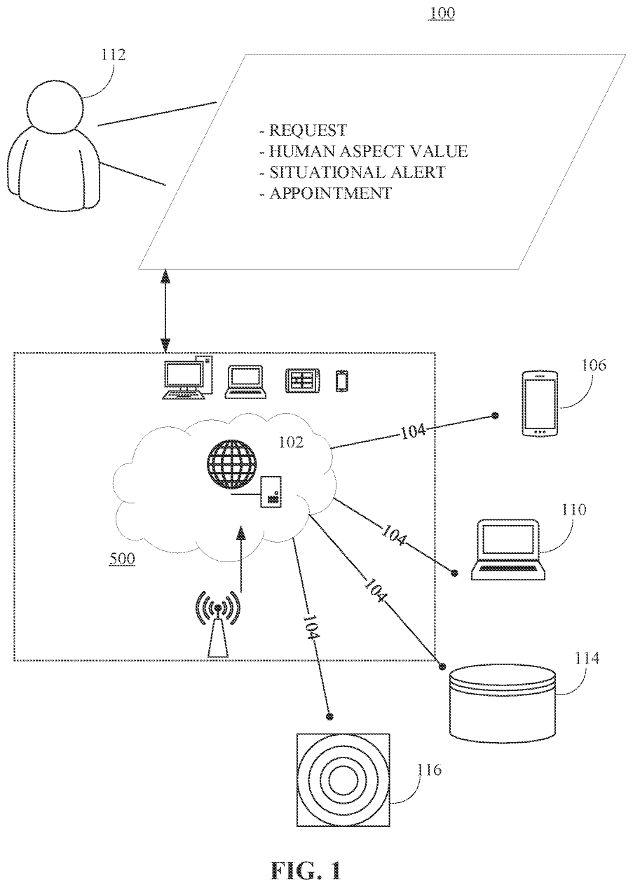

Referring now to figures, FIG. 1 is an illustration of an online platform 100 consistent with various embodiments of the present disclosure. By way of non-limiting example, the online platform 100 for facilitating assessment of well-being of a user based on feedback may be hosted on a centralized server 102, such as, for example, a cloud computing service. The centralized server 102 may communicate with other network entities, such as, for example, a mobile device 106 (such as a smartphone, a laptop, a tablet computer, etc.), other electronic devices 110 (such as desktop computers, server computers, etc.), databases 114, and sensors 116 over a communication network 104, such as, but not limited to, the Internet. Further, users of the online platform 100 may include relevant parties such as, but not limited to, end-users, service providers, managers, and administrators. Accordingly, in some instances, electronic devices operated by the one or more relevant parties may be in communication with the online platform 100.

A user 112, such as the one or more relevant parties, may access the online platform 100 through a web-based software application or browser. The web-based software application may be embodied as, for example, but not be limited to, a website, a web application, a desktop application, and a mobile application compatible with a computing device 500.

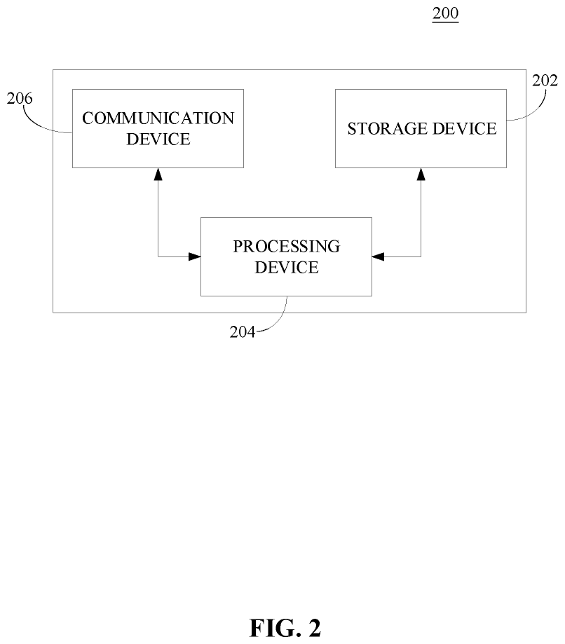

FIG. 2 is a block diagram 200 of a system for facilitating assessment of well-being of a user based on feedback, in accordance with some embodiments. Accordingly, the system may include a communication device 206 configured for receiving a request from at least one first user device associated with at least one first user. Further, the at least one first user may include an individual, an institution, and an organization. Further, the at least one first user device may include a smartphone, a tablet, a laptop, a personal computer, and so on. Further, the request may correspond to the at least one first user that may want to compute human aspect value of at least one second user. Further, the at least one second user may include an individual, an institution, and an organization. Further, the communication device 206 may be configured for transmitting a questionnaire to at least one second user device. Further, the at least one second user device may include a smartphone, a tablet, a laptop, a personal computer, and so on. Further, the communication device may be configured for receiving at least one response associated with a time interval from the at least one second user device, wherein the at least one response is associated with the questionnaire. Further, the time interval may include a week, a month, a year, etc. Further, the communication device 206 may be configured for transmitting a human aspect value and a graphical representation to at least one of the at least one first user device and the at least one second user device.

Further, the system may include a processing device 204 configured for analyzing the at least one response, the questionnaire based on customizable individual coefficients. Further, the processing device 204 may be configured for generating the graphical representation based on the analyzing. Further, the graphical representation may include average weighted values plotted in relation to a status scale from 1-5. Further, the processing device may be configured for processing a participation value, the customizable individual coefficients, a predetermined coefficient, the at least one response, and the questionnaire. Further, the processing device 204 may be configured for determining the human aspect value associated with the at least one second user based on the processing.

Further, the system may include a storage device 202 configured for retrieving the questionnaire from a blockchain. Further, the questionnaire may be associated with human categories (or aspects) that may include mind, body, social, environment, etc. Further, the human categories may be customizable. Further, the blockchain may facilitate preventing malicious attacks from unauthorized users such as hackers. Further, the storage device 202 may be configured for storing the at least one response on the blockchain. Further, the storage device 202 may be configured for retrieving the customizable individual coefficients (or weights) corresponding to the human categories of the questionnaire from the blockchain. Further, the storage device 202 may be configured for storing the human aspect value and the graphical representation on the blockchain. Further, the storage device 202 may be configured for retrieving the participation value and the predetermined coefficient from the blockchain.

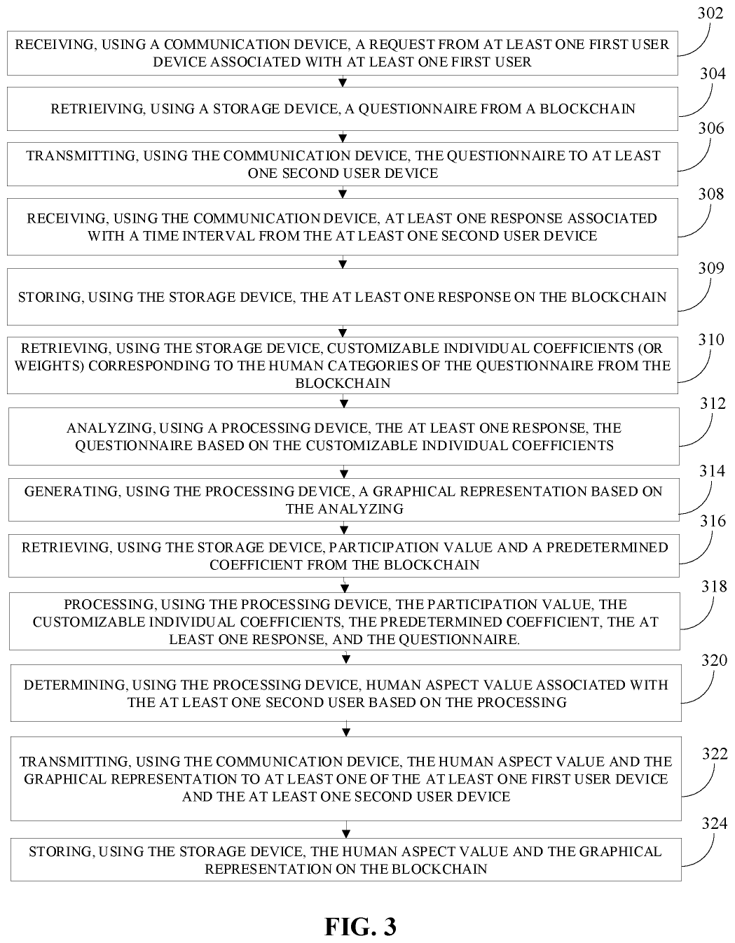

FIG. 3 is a flow chart of a method for facilitating assessment of well-being of a user based on feedback, in accordance with some embodiments. Accordingly, the method may include receiving, using a communication device, a request from at least one first user device associated with at least one first user 302. Further, the at least one first user may include an individual, an institution, and an organization. Further, the at least one first user device may include a smartphone, a tablet, a laptop, a personal computer, and so on. Further, the request may correspond to the at least one first user that may want to compute assess the well-being of at least one second user. Further, the at least one second user may include an individual, an institution, and an organization. Further, in an embodiment, the at least one first user may include a senior (such as a manager) to the at least one second user (or an employee) in an organization (or company).

Further, the method may include retrieving, using a storage device, a questionnaire from a blockchain 304. Further, the questionnaire may be associated with human categories (or aspects) that may include mind, body, social, environment, etc. Further, the human categories may be customizable. Further, the blockchain may facilitate preventing malicious attacks from unauthorized users such as hackers.

Further, the method may include transmitting, using the communication device, the questionnaire to at least one second user device 306. Further, the at least one second user device may include a smartphone, a tablet, a laptop, a personal computer, and so on.

Further, the method may include receiving, using the communication device, at least one response associated with a time interval from the at least one second user device 308, wherein the at least one response is associated with the questionnaire. Further, the time interval may include a week, a month, a year, etc.

Further, the method may include storing, using the storage device, the at least one response on the blockchain 309.

Further, the method may include retrieving, using the storage device, customizable individual coefficients (or weights) corresponding to the human categories of the questionnaire from the blockchain 310.

Further, the method may include analyzing, using a processing device, the at least one response, the questionnaire based on the customizable individual coefficients 312.

Further, the method may include generating, using the processing device, a graphical representation based on the analyzing 314. Further, the graphical representation may include average weighted values plotted in relation to a status scale from 1-5.

Further, the method may include retrieving, using the storage device, a participation value and a predetermined coefficient from the blockchain 316.

Further, the method may include processing, using the processing device, the participation value, the customizable individual coefficients, the predetermined coefficient, the at least one response, and the questionnaire 318.

Further, the method may include determining, using the processing device, human aspect value associated with the at least one second user based on the processing 320.

Further, the method may include transmitting, using the communication device, the human aspect value and the graphical representation to at least one of the at least one first user device and the at least one second user device 322.

Further, the method may include storing, using the storage device, the human aspect value and the graphical representation on the blockchain 324.

Further, in some embodiments, the method may include receiving, using the communication device, sensor data from at least one sensor. Further, the at least one sensor may be disposed on the at least one second user device. Further, the at least one second user device may include a smartwatch, a smart goggle, etc. Further, the at least one sensor may include a physiological sensor, an audio sensor, an image sensor, a location sensor, etc. Further, in an embodiment, the at least one sensor may be disposed in a locality associated with the at least one second user. Further, the physiological sensor may be configured for detecting physiological parameters associated with the at least one second user. Further, in an embodiment, the physiological sensor may be disposed on a body of the at least one second user. Further, the physiological parameters may include heartbeat, pulse rate, blood pressure, body temperature, etc. Further, the method may include retrieving, using the storage device, a machine learning model from the blockchain. Further, the machine learning model may be configured for detecting physical correlates associated with the at least one second user. Further, the physical correlates may facilitate identifying characteristic identifiers. Further, the characteristic identifiers may be associated with a state of the human categories. Further, the state may include sentiments such as happiness, grief, amazement, etc. Further, the method may include analyzing, using the processing device, the sensor data based on the machine learning model. Further, the method may include generating, using the processing device, a report based on the analyzing of the sensor data. Further, the report may include the characteristic identifiers. Further, the method may include transmitting, using the communication device, the report to at least one of the at least one second user device and the at least one first user device. Further, the method may include storing, using the storage device, the report on the blockchain. Further, the generating of the human aspect value may be based on the analyzing of the sensor data.

Further, in some embodiments, the method may include generating, using the processing device, a situational alert based on the analyzing of the sensor data. Further, the method may include transmitting, using the communication device, the situational alert to the at least one first user device. Further, the situational alert may prompt the at least one first user to schedule an appointment with the at least one second user that may be facing well-being issues such as social separation, health issues, mental issues, financial issues, etc. Further, the method may include receiving, using the communication device, an appointment prompt from the at least one first user device. Further, the method may include generating, using the processing device, an appointment corresponding to the receiving of the appointment prompt. Further, the method may include transmitting, using the communication device, the appointment to the at least one second user device. Further, based on the appointment, the at least one first user may assist the at least one second user facing the well-being issues. Further, the appointment may include a voice call, a video call, a meeting schedule, etc.

Further, in some embodiments, the method may include generating, using the processing device, a cross-validation alert based on the analyzing of the sensor data. Further, the cross-validation alert may include a positive cross-validation alert and a negative cross-validation alert. Further, the positive cross-validation alert may facilitate validation of the at least one response. Further, the negative cross-validation alert may facilitate rejection of validation (or authenticity) of the at least one response. Further, the method may include transmitting, using the communication device, the cross-validation alert to at least one of the at least one first user device and the at least one second user device.

Further, in some embodiments, the method may include receiving, using the communication device, biometric data associated with the at least one second user from a biometric sensor. Further, the biometric sensor may include an iris sensor, a fingerprint sensor, etc. Further, the biometric sensor may be configured for detecting an identity of the at least one second user. Further, the method may include analyzing, using the processing device, the biometric data to generate a validation alert associated with the at least one second user. Further, the validation alert may include a positive alert and a negative alert. Further, the positive alert may correspond to true identity of the at least one second user. Further, the negative alert may correspond to false identity of the at least one second user. Further, the validation alert may facilitate preventing fraud and proxy attempts by the unauthorized users such as hackers or third-party users. Further, the method may include transmitting, using the communication device, the validation alert to the at least one first user device. Further, the method may include storing, using the storage device, the validation alert to the blockchain. Further, the transmitting of the questionnaire may be based on the validation alert.

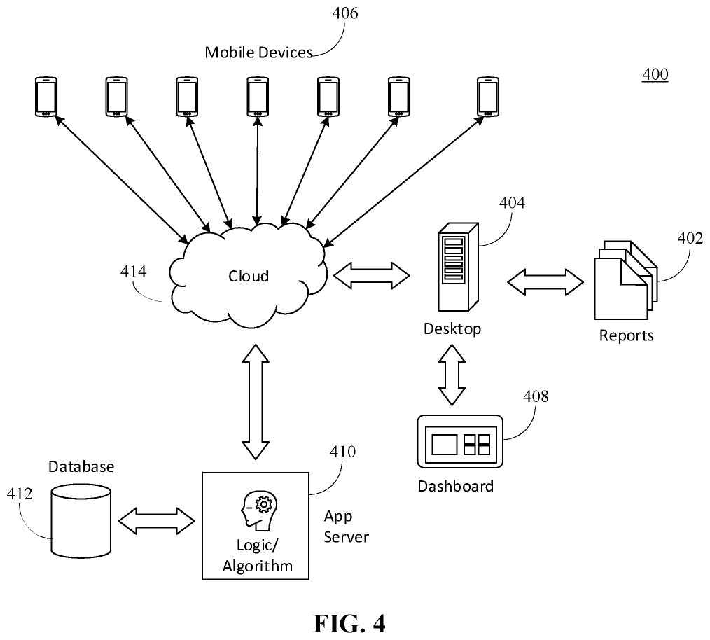

FIG. 4 is a schematic of a system 400 for facilitating the assessment of the well-being of a user based on feedback, in accordance with some embodiments. Accordingly, the system may include a plurality of user devices associated with a plurality of users 406. Further, the plurality of user devices may be communicatively coupled with cloud storage 414. Further, the cloud storage may be communicatively coupled to desktops associated with the plurality of users 404. Further, the desktops may be used to access reports (such as the human aspect value) 402. Further, the cloud storage may include logic algorithms associated with an application server of a software platform 410. Further, the cloud storage may be communicatively coupled with a database 412.

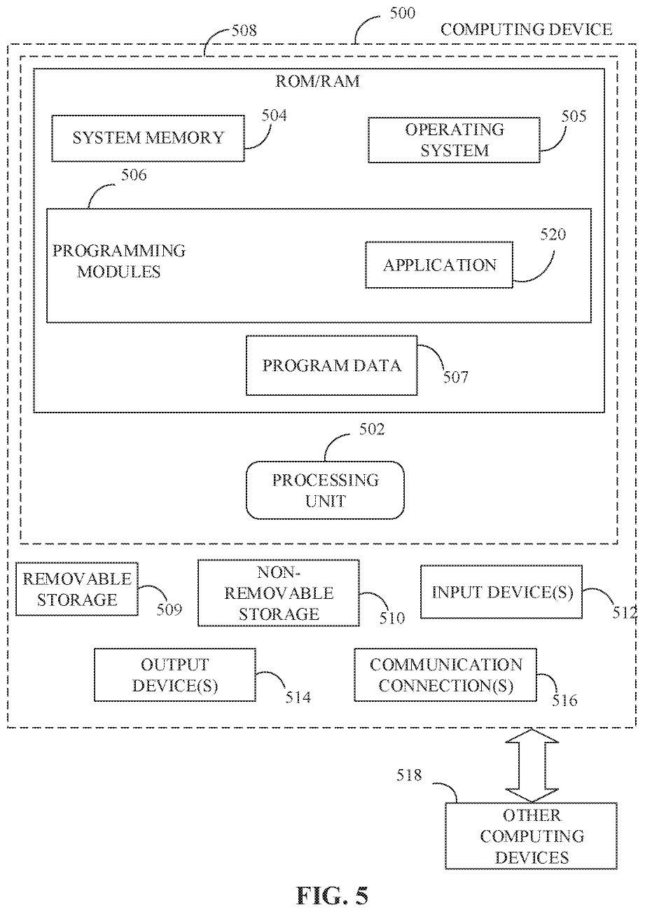

With reference to FIG. 5, a system consistent with an embodiment of the disclosure may include a computing device or cloud service, such as computing device 500. In a basic configuration, computing device 500 may include at least one processing unit 502 and a system memory 504. Depending on the configuration and type of computing device, system memory 504 may comprise, but is not limited to, volatile (e.g. random-access memory (RAM)), non-volatile (e.g. read-only memory (ROM)), flash memory, or any combination. System memory 504 may include operating system 505, one or more programming modules 506, and may include a program data 507. Operating system 505, for example, may be suitable for controlling computing device 500's operation. In one embodiment, programming modules 506 may include an image-processing module, machine learning module, and/or image classifying module. Furthermore, embodiments of the disclosure may be practiced in conjunction with a graphics library, other operating systems, or any other application program and is not limited to any particular application or system. This basic configuration is illustrated in FIG. 5 by those components within a dashed line 508.

Computing device 500 may have additional features or functionality. For example, computing device 500 may also include additional data storage devices (removable and/or non-removable) such as, for example, magnetic disks, optical disks, or tape. Such additional storage is illustrated in FIG. 5 by a removable storage 509 and a non-removable storage 510. Computer storage media may include volatile and nonvolatile, removable, and non-removable media implemented in any method or technology for storage of information, such as computer-readable instructions, data structures, program modules, or other data. System memory 504, removable storage 509, and non-removable storage 510 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 500. Any such computer storage media may be part of device 500. Computing device 500 may also have input device(s) 512 such as a keyboard, a mouse, a pen, a sound input device, a touch input device, a location sensor, a camera, a biometric sensor, etc. Output device(s) 514 such as a display, speakers, a printer, etc. may also be included. The aforementioned devices are examples and others may be used.

Computing device 500 may also contain a communication connection 516 that may allow device 500 to communicate with other computing devices 518, such as over a network in a distributed computing environment, for example, an intranet or the Internet. Communication connection 516 is one example of communication media. Communication media may typically be embodied by computer-readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer-readable media as used herein may include both storage media and communication media.

As stated above, a number of program modules and data files may be stored in system memory 504, including operating system 505. While executing on processing unit 502, programming modules 506 (e.g., application 520 such as a media player) may perform processes including, for example, one or more stages of methods, algorithms, systems, applications, servers, databases as described above. The aforementioned process is an example, and processing unit 502 may perform other processes. Other programming modules that may be used in accordance with embodiments of the present disclosure may include sound encoding/decoding applications, machine learning application, acoustic classifiers, etc.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general-purpose graphics processor-based systems, multiprocessor systems, microprocessor-based or programmable consumer electronics, application-specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer-readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid-state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the invention has been explained in relation to its preferred embodiment, it is to be understood that many other possible modifications and variations can be made without departing from the spirit and scope of the invention.

What is claimed is:

1. A system for facilitating assessment of well-being of a user based on feedback, comprising:
a communication device configured for receiving a request from at least one first user device associated with at least one first user;
the communication device configured for transmitting a human aspect value and a graphical representation to at least one of the at least one first user device and at least one second user device;
a processing device configured for analyzing an at least one response and a questionnaire based on customizable individual coefficients;
the processing device configured for generating the graphical representation based on the analyzing;
the processing device configured for processing a participation value, the customizable individual coefficients, a predetermined coefficient, the at least one response, and the questionnaire;
the processing device configured for determining the human aspect value associated with the at least one second user;
a storage device configured for retrieving the questionnaire from a blockchain;
the storage device retrieving the customizable individual coefficients corresponding to human categories of the questionnaire from the blockchain; and
the storage device configured for storing the human aspect value and a graphical representation on the blockchain.

2. The system for facilitating assessment of well-being of a user based on feedback of claim 1, wherein the communication devices is configured for:
transmitting a questionnaire to at least one second user device; and
receiving at least one response associated with a time interval from the at least one second user device, wherein the at least one response is associated with the questionnaire.

3. The system for facilitating assessment of well-being of a user based on feedback of claim 1, wherein the processing device is configured for generating automated prompts to the user device based on the analyzing.

4. The system for facilitating assessment of well-being of a user based on feedback of claim 1, wherein the storage device is configured for:
retrieving the participation value and the predetermined coefficient from the blockchain;
storing the at least one response on the blockchain;
retrieving the customizable individual coefficients corresponding to the human categories of the questionnaire from the blockchain; and
retrieving the participation value and the predetermined coefficient from the blockchain.

5. The system for facilitating assessment of well-being of a user based on feedback of claim 1, wherein the processing device is configured for:
generating a human aspect value; and
the human aspect value is the sum of the Aspect Value and the Participation (Engagement) Value multiplied by predefined coefficients.

6. The system for facilitating assessment of well-being of a user based on feedback of claim 1, wherein communication device is configured for transmitting the questionnaire wherein the answers correspond to a predefined value range.

7. The system for facilitating assessment of well-being of a user based on feedback of claim 1, wherein the processing device is configured for:
generating values collected from the questionnaire; and
the values collected from the questionnaire are then averaged using predefined customizable individual coefficients or weights.

8. The system for facilitating assessment of well-being of a user based on feedback of claim 5, wherein the processing device is configured for:
generating a participation value; and
the Participation Value is compared to the average participation value of all other users.

9. The system for facilitating assessment of well-being of a user based on feedback of claim 5, wherein the processing device is configured for:
generating an Aspect Value; and
the Aspect Value relates to mind, body, social, environment, and other predefined characteristics.

10. A method for facilitating assessment of well-being of a user based on feedback, comprising;
receiving, using a communication device, a request from at least one first user device associated with at least one first user;
retrieving, using a storage device, a questionnaire from a blockchain;
transmitting, using the communication device, the questionnaire to at least one second user device;
receiving, using the communication device, at least one response associated with a time interval from the at least one second user device, wherein the at least one response is associated with the questionnaire;
storing, using the storage device, the at least one response on the blockchain;
retrieving, using the storage device, customizable individual coefficients corresponding to the human categories of the questionnaire from the blockchain;
analyzing, using a processing device, the at least one response, the questionnaire based on the customizable individual coefficients;
generating, using the processing device, a graphical representation based on the analyzing;
retrieving, using the storage device, a participation value and a predetermined coefficient from the blockchain;
processing, using the processing device, the participation value, the customizable individual coefficients, the predetermined coefficient, the at least one response, and the questionnaire;
determining, using the processing device, human aspect value associated with the at least one second user based on the processing;
transmitting, using the communication device, the human aspect value and the graphical representation to at least one of the at least one first user device and the at least one second user device; and
storing, using the storage device, the human aspect value and the graphical representation on the blockchain.

11. The method for facilitating assessment of well-being of a user based on feedback of claim 10, wherein the processing device is configured for generating automated prompts to the user device based on the analyzing.

12. The method for facilitating assessment of well-being of a user based on feedback of claim 10, wherein the processing device is configured for:
generating a human aspect value; and
the human aspect value is the sum of the Aspect Value and the Participation (Engagement) Value multiplied by predefined coefficients.

13. The method for facilitating assessment of well-being of a user based on feedback of claim 10, wherein communication device is configured for transmitting the questionnaire wherein the answers correspond to a predefined value range.

14. The method for facilitating assessment of well-being of a user based on feedback of claim 10, wherein the processing device is configured for:
generating values collected from the questionnaire; and
the values collected from the questionnaire are then averaged using predefined customizable individual coefficients or weights.

15. The method for facilitating assessment of well-being of a user based on feedback of claim 12, wherein the processing device is configured for:
generating a participation value; and
the Participation Value is compared to the average participation value of all other users.

16. The method for facilitating assessment of well-being of a user based on feedback of claim 12, wherein the processing device is configured for:
generating an Aspect Value; and
the Aspect Value relates to mind, body, social, environment, and other predefined characteristics.
